(19) United States
(12) Patent Application Publication
(10) Pub. No.: US 2006/0046276 A1
(43) Pub. Date: Mar. 2, 2006

(54) Acetyl-LDL receptor as a biomarker for breast cancer

(75) Inventors: Ira L. Goldknopf, The Woodlands, TX (US); Essam A. Sheta, The Woodlands, TX (US)

Correspondence Address: ELIZABETH R. HALL, 1722 MARYLAND STREET, HOUSTON, TX, 77006, US

(73) Assignee: Power3 Medical Products, Inc., The Woodlands, TX (US)

(21) Appl. No.: 10/931,333

(22) Filed: Sep. 1, 2004

(51) Int. Cl. G01N 33/567; G01N 33/574

(52) U.S. Cl. 435/7.21; 435/7.23

(57) ABSTRACT

The present invention is a biomarker for breast cancer, including early stage and precancerous conditions. Patients exhibiting reduced expression of acetyl-LDL receptor in a NAF sample collected from a breast that is below the 95% confidence limit of normals either have breast cancer are at high risk to develop breast cancer and should become the object of increased medical surveillance.

### BACKGROUND OF THE INVENTION

1. Field of the Invention

The invention relates to the identification of a biomarker for the early detection of breast disease. More particularly, the present invention relates to the identification of the acetyl-LDL receptor as a biomarker useful for both the early detection of breast cancer, as an indicator of risk for the development of breast cancer, and in the treatment of breast cancer.

2. Description of the Related Art

Proteonomics is a new field of medical research wherein proteins are identified and linked to biological functions, including roles in a variety of disease states. With the completion of the mapping of the human genome, the identification of unique gene products, or proteins, has increased exponentially. In addition, molecular diagnostic testing for the presence of certain proteins already known to be involved in certain biological functions has progressed from research applications alone to use in disease screening and diagnosis for clinicians. However, proteonomic testing for diagnostic purposes remains in its infancy. There is, however, a great deal of interest in using proteonomics for the elucidation of potential disease biomarkers.

Detection of abnormalities in the genome of an individual can reveal the risk or potential risk for individuals to develop a disease. The transition from risk to emergence of disease can be characterized as an expression of genomic abnormalities in the proteome. Thus the appearance of abnormalities in the proteome signals the beginning of the process of cascading effects that can result in the deterioration of the health of the patient. Therefore, detection of proteomic abnormalities at an early stage is desired in order to allow for detection of disease either before it is established or in its earliest stages where treatment may be effective.

Recent progress using a novel form of mass spectrometry called surface enhanced laser desorption and ionization time of flight (SELDI-TOF) for the testing of ovarian cancer has led to an increased interest in proteonomics as a diagnostic tool (Petrocoin, E. F. et al. 2002. Lancet 359:572-577). Furthermore, proteonomics has been applied to the study of breast cancer through use of 2D gel electrophoresis and image analysis to study the development and progression of breast carcinoma in patients (Kuerer, H. M. et al. 2002. Cancer 95:2276-2282).

In the case of breast cancer, breast ductal fluid specimens have been used to identify distinct protein expression patterns in bilateral matched pair ductal fluid samples of women with unilateral invasive breast carcinoma. This method of diagnosing and monitoring breast cancer was detailed in U.S. patent application Ser. No. 10/236,027 filed Apr. 24, 2003, and patent application Ser. No. 10/301,512 filed Nov. 20, 2002 where a side-by-side comparison was used to determine differences in protein expression profiles between cancerous breasts and those free of cancer.

In spite of widespread mammographic screening for over twenty years, the likelihood of dying from breast cancer has been reduced only slightly. Over 200,000 women will be diagnosed with invasive breast cancer this year, and nearly 60,000 additional women will be diagnosed with in situ (early) cancer. But for the 45,000 women who die every year from this disease, mammographic screening as a public health policy has been a failure.

Granted, less than half of eligible women get mammograms regularly, some never do, and some women are “too young” to start according to current guidelines, but not too young to develop breast cancer. However, even if all women followed guidelines exactly, the mortality rate of breast cancer would at best only be cut by one-third. While this “ideal scenario” would far surpass any benefit seen during the past two decades, 30,000 breast cancer patients would still die each year.

Since early diagnosis is the key to surviving breast cancer, identification of disease biomarkers has been an active research area. Genetic screening using individual biomarker genes, such as BRCA 1 and BRCA 2, or proteins, such as the HER-2/nu, have improved the screening of breast cancer for potential sensitivity to treatment agents such as Herceptin (Hayes, D. F. et al. 2001. Clin. Cancer Res. 7:2601-2604). Unfortunately, a low percentage of breast cancers are found positive for such cancer-related genes.

This problem is underscored by the fact that these genomic tests are the primary way of screening in pre-menopausal patients (Bradbury, J. 2002. Lancet Oncol. 3:2). Further, standard estrogen and progesterone receptor tests, which require a biopsy of the tumor, and other similar combinations of diagnostics, have improved the predictability of breast cancer survival by only a small percentage (Molino, A. et al. 1997. Breast Cancer Res. Treat. 45:241-249).

Analysis of the biochemical and cellular contents of breast ductal fluid has been of recent interest to researchers attempting to identify disease biomarkers. In one study, the authors reported the identification of over 1000 distinct proteins expressed in bilateral matched pair breast ductal fluid specimens from women with unilateral invasive breast carcinoma (Kuerer, H. M. et al. 2002. Cancer 95:2276-2282). The researchers used two dimensional (2D) polyacrylamide gel electrophoresis and nipple aspirate fluid samples and determined from the side-by-side comparison of the gels that there were qualitative differences in protein expression. They found that proteins were differentially expressed in the nipple aspirate fluid (NAF) from contra lateral breasts where cancer had been detected when compared to NAF samples from contra lateral breasts that had been determined to be free of cancer.

Using a different method for proteonomic analysis, SELDI-TOF, investigators have generated proteomic spectra from serum samples of ovarian cancer patients (Petrocoin, E. F. et al. 2002. Lancet 359:572-577) and from nipple aspirate fluid samples (Paweletz, C. P. et al. 2001). Dis. Markers 17:301-307). Since SELDI-TOF only separates small proteins on the basis of molecular weight, however, it lacks the scope and separation power of 2D gel electrophoresis, a method where all sizes of proteins are separated by both isoelectric focusing according to a protein's isoelectric point and by molecular weight.

There remains a need for better ways to detect and diagnose breast cancer, including a need for specific biomarkers of the disease. An additional need exists for improved methods and compositions for the treatment of breast cancer.

### SUMMARY OF THE INVENTION

The present invention relates to acetyl-LDL receptor as a biomarker for breast disease, particularly breast cancer. One aspect of the present invention provides a sensitive method for early detection and diagnosis of breast cancer by assessing the acetyl-LDL receptor concentration in breast nipple aspirate fluid. An acetyl-LDL receptor concentration in nipple aspirate fluid collected from a patient's breast that is significantly below the acetyl-LDL receptor concentration of nipple aspirate fluid samples from normal breasts indicates a strong likelihood of breast cancer or a pre-cancerous condition in the breast having the reduced expression of acetyl-LDL receptor.

Another aspect of the invention is the assessment of the risk of developing breast cancer by comparing the acetyl-LDL receptor levels in nipple aspirate fluid samples collected from the right and left breast of a patient. When the two breasts exhibit widely divergent levels of acetyl-LDL receptor and one breast has lower levels than normal breasts, the patient is considered at high-risk for the development of breast disease, including breast cancer and should seek further diagnosis of breast disease. The present invention also includes a method of using nipple aspirate fluid to diagnose breast cancer, the method comprising: (a) collecting a nipple aspirate fluid sample from a test subject; analyzing the nipple aspirate fluid sample for a reduced expression of acetyl-LDL receptor protein; and using the expression of acetyl-LDL receptor protein to diagnose the test subject.

Yet another aspect of the invention is a method for screening for breast cancer comprising: obtaining a breast ductal secretion from a patient's breast; determining a quantity of an acetyl-LDL receptor protein in the patient's breast ductal secretion; and comparing the quantity of the acetyl-LDL receptor protein in the patient's breast ductal secretion with a normal value of acetyl-LDL receptor protein in breast ductal fluid from control breasts; whereby a reduction in the quantity of the acetyl-LDL receptor protein in the patient's breast ductal secretion to a level less than the normal value of acetyl-LDL receptor protein in control breasts is indicative of a cancerous or a pre-cancerous condition in the patient's breast.

Still yet another aspect of the invention is A method for diagnosing breast cancer comprising: obtaining a breast ductal secretion from two breasts of a subject; determining a quantity of an acetyl-LDL receptor protein in the breast ductal secretion of each of the two breasts; and comparing a concentration of acetyl-LDL receptor protein in breast ductal fluid from control breasts with the lower quantity of the acetyl-LDL receptor protein from the patient's two breasts. A cancerous or pre-cancerous condition in the patient's breast having the lower quantity of acetyl-LDL receptor protein is indicated whenever the lower quantity of acetyl-LDL receptor protein is at least 50% less than the quantity of acetyl-LDL receptor protein in the ductal secretion of the other breast and is equal to or less than the mean plus one standard deviation of acetyl-LDL receptor protein concentration in control breasts.

Another aspect of the invention is a method for diagnosing breast cancer comprising: obtaining a nipple aspirate fluid sample from a breast; separating a protein fraction of the nipple aspirate fluid sample by two-dimensional gel electrophoresis; determining a quantity of an acetyl-LDL receptor protein in the nipple aspirate fluid sample; and using the quantity of acetyl-LDL receptor protein to diagnose breast cancer in the breast.

A further aspect of the invention is a method for diagnosing breast cancer comprising: obtaining a breast ductal secretion from a breast; determining a quantity of an acetyl-LDL receptor protein in the breast ductal secretion using an antibody reactive with an antigenic determinant in an acetyl-LDL receptor protein; and using the quantity of acetyl-LDL receptor protein to diagnose breast cancer in the breast.

The foregoing has outlined rather broadly several aspects of the present invention in order that the detailed description of the invention that follows may be better understood. Additional features and advantages of the invention will be described hereinafter which form the subject of the claims of the invention. It should be appreciated by those skilled in the art that the conception and the specific embodiment disclosed might be readily utilized as a basis for modifying or redesigning the structures for carrying out the same purposes as the invention. It should be realized by those skilled in the art that such equivalent constructions do not depart from the spirit and scope of the invention as set forth in the appended claims.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1 illustrates the results of a 2D gel image analysis of a nipple aspirate fluid sample of a normal breast with the three acetyl-LDL receptor spots marked.

FIG. 2 is a graph indicating the acetyl-LDL receptor concentrations of each breast of four normal women, two high-risk women, and twelve women diagnosed with unilateral breast cancer.

### DESCRIPTION OF THE PREFERRED EMBODIMENTS

The present invention relates to a biomarker for breast tissue disease. More particularly, the present invention relates to the identification of the acetyl-LDL receptor as a biomarker useful for both the early recognition of breast cancer and a high-risk for the development of breast cancer.

The method for identification of acetyl-LDL receptor as a biomarker for breast disease, particularly breast cancer, is based on the comparison of 2D gel electrophoretic images of breast ductal fluid obtained by breast nipple aspiration or ductal lavage from women with and without diagnosed breast cancer.

2D gel electrophoresis has been used in research laboratories for biomarker discovery since the 1970's (Goldknopf, I. L. et al. 1977. Proc. Natl. Acad. Sci. USA 74:864-868). In the past, this method has been considered highly specialized, labor intensive and non-reproducible. Only recently with the advent of integrated supplies, robotics, and software combined with bioinformatics has progression of this proteonomics technique in the direction of diagnostics become feasible. The promise and utility of 2D gel electrophoresis is based in its ability to detect changes in protein expression and to discriminate protein isoforms that arise due to variations in amino acid sequence and/or post-synthetic protein modifications such as phosphorylation, ubiquitination, conjugation with ubiquitin-like proteins, acetylation, and glycosylation. These are important variables in cell regulatory processes involved in cancer and other diseases.

There are few comparable alternatives to 2D gels for tracking changes in protein expression patterns related to disease progression. The introduction of high sensitivity fluorescent staining, digital image processing and computerized image analysis has greatly amplified and simplified the detection of unique species and the quantification of proteins. By using known protein standards as landmarks within each gel run, computerized analysis can detect unique differences in protein expression and modifications between two samples from the same individual or between several individuals.

Proteins of interest can be excised from the gels and the proteins can then be identified by in gel digestion and matrix assisted laser desorption time of flight mass spectroscopy (MALDI-TOF MS) based peptide mass fingerprinting and database searching or liquid chromatography with tandem mass spectrometry partial sequencing of individual peptides (LCMS/MS).

The identification of the acetyl-LDL receptor as a biomarker of breast disease was based on a comparison of the 2D gel electrophoretic images of multiple samples of breast ductal fluid, obtained by breast nipple aspiration, from women with and without diagnosed breast cancer.

Analysis of the contents of breast ductal fluid has recently gained attention as a potential non-invasive method for studying the local microenvironment associated with development and progression of breast carcinoma (Kuerer, H. M. et al. 2002. Cancer 95:2276-2282; Doley, W. et al. 2001. J. Natl. Cancer. Inst. 93:1624-1632; Wrensch, M. R. et al. 2001. J. Natl. Cancer Inst. 93:1791-1798). In most breast cancers, the sites of disease origin are the ductal or lobular epithelial cells of the breast, which secrete into the ducts. Only a fraction of the breast secretion is needed to study the protein concentrations. As little as one to two microliters of fluid, obtained through nipple aspiration, is sufficient.

Research has shown that the use of cytological analysis of breast nipple aspirate fluid and/or fluid obtained by ductal lavage is successful as a predictor of cancer risk (Doley, W. et al. 2001. J. Natl. Cancer. Inst. 93:1624-1632; Wrensch, M. R. et al. 2001. J. Natl. Cancer Inst. 93:1791-1798). The U.S. Food and Drug Administration (FDA) and Blue Cross Blue Shield Insurance Company have approved cytological tests using either type of fluid (Doley, W. et al. 2001. J. Natl. Cancer. Inst. 93:1624-1632; Wrensch, M. R. et al. 2001. J. Natl. Cancer Inst. 93:1791-1798).

Therefore, experiments were performed using breast ductal fluid samples collected non-invasively by nipple aspiration. The samples were taken from both breasts of 12 unilateral breast cancer patients, 4 control or normal women with no known breast disease, and two mammogram negative women with a history of breast cancer in their family and where onset of disease had begun at the same age as their age when the samples were taken.

Sample Collection and Preparation

Sample collection and storage may be performed in many different ways depending on the type of sample and the conditions of the collection process. One of skill in the art would apply sample collection techniques well known in the art. The nipple aspirate fluid (NAF) samples were collected for the study detailed herein using a simple, non-invasive, suction device similar to a manual breast pump. However, needle biopsy cores, surgical resection samples, lymph node tissue and other breast related samples could be used.

NAF samples were prepared for protein analysis by first washing with trichloroacetic acid (TCA) followed by two washes with acetone. This washing allowed for greater sensitivity and resolution for protein separation in the nipple aspirate fluid as compared to previous sample preparation methods, with more than 1200 distinctive proteins detected as opposed to 60-65 poorly resolved proteins obtained previously.

Each collected NAF sample was first diluted with the addition of cold buffer (e.g., isotonic saline, Tris HCl, RPMI and the like) containing a mixture of protease inhibitors (e.g., PMSF, leupeptin, pepstatin, chymostatin, calpain inhibitor I, calpain inhibitor II, EDTA-free protease inhibitor cocktail, and the like). Preferably, each sample was diluted with the addition of cold RPMI buffer containing an EDTA-free protease inhibitor cocktail. The diluted nipple aspirate fluid (NAF) was aliquoted into 1.5 ml microfuge tubes in 100 μl portions and frozen in liquid nitrogen before analysis.

In a preferred embodiment of the invention, NAF samples containing the protease inhibitor cocktail are taken from −80° C. and placed on ice for thawing. To each 100 μl of sample, 100 μl of LB-1 buffer (7M urea, 2M Thiourea, 1% DTT, 1% Triton X-100, 1X Protease inhibitors, and 0.5% Ampholyte pH 3-10) was added and the mixture vortexed. The sample was incubated at room temperature for about 5 minutes.

Two Dimensional-Electrophoresis of Samples

Separation of the proteins in nipple aspirate fluid was then performed using 2D gel electrophoresis. The 2D gel electrophoretic images were obtained, compared and analyzed as described in the U.S. Provisional Patent Application Ser. No. 60/591,312 entitled “Differential Protein Expression Patterns Related to Disease States” filed Jul. 27, 2004 and incorporated herein by reference.

After the NAF sample had been incubated with the LB-1 buffer, 300 μl UPPA-I (Perfect Focus, Genotech) was added to each sample and the sample vortexed and incubated on ice for 15 minutes. Next 300 μl UPPA-II (Perfect Focus, Genotech) was added to each tube, vortexed and centrifuged at about 15,000×g for 5 minutes at 4° C. The entire supernatant was carefully removed by vacuum aspiration. Repeat centrifugation at about 15,000×g for 30 seconds was performed. The remaining supernatant was removed by vacuum aspiration.

The pellet was suspended in 25 μl of Ultra Pure H₂O and vortexed. Then 1 ml of OrgoSol (Perfect Focus, Genotech, prechilled at −20° C.) and 5 μl SEED (Perfect Focus, Genotech) were added to each pellet and incubated at −20° C. for about 30 minutes. The pellet was suspended using repeated vortexing bursts of about 20-30 seconds each. The tubes were then centrifuged at about 15,000×g for 5 minutes. The entire supernatant was carefully removed by vacuum aspiration. The water suspension and the OrgoSol-SEED wash of the pellet were repeated.

The protein pellet was air dried for about 5 minutes, then the pellet was dissolved in an appropriate amount of isoelectric focusing (IEF) loading buffer, incubated at room temperature and vortexed periodically until the pellet was dissolved to visual clarity. The samples were centrifuged briefly before a protein assay was performed on the sample.

An aliquot of 100 μg of NAF proteins was suspended in a total volume of 184 μl of IEF loading buffer and 1 μl Bromophenol Blue. Each sample was loaded onto an 11 cm IEF strip (Bio-Rad), pH 4-7, and overlaid with 1.5-3.0 ml of mineral oil to minimize the sample buffer evaporation. Using the PROTEAN® IEF Cell, an active rehydration was performed at 50V and 20° C. for 12-18 hours.

IEF strips were then transferred to a new tray and focused for 20 min at 250V followed by a linear voltage increase to 8000V over 2.5 hours. A final rapid focusing was performed at 8000V until 20,000 volt-hours were achieved. Running the IEF strip at 500V until the strips were removed finished the isoelectric focusing process.

Isoelectric focused strips were incubated on an orbital shaker for 15 min with equilibration buffer (2.5 ml buffer/strip). The equilibration buffer contained 6M urea, 2% SDS, 0.375M HCl, and 20% glycerol, as well as freshly added DTT to a final concentration of 30 mg/ml. An additional 15 min incubation of the IEF strips in the equilibration buffer was performed as before, except freshly added iodoacetamide (C₂H₄INO) was added to a final concentration of 40 mg/ml. The IPG strips were then removed from the tray using clean forceps and washed five times in a graduated cylinder containing the Bio Rad running buffer 1X Tris-Glycine-SDS.

The washed IEF strips were then laid on the surface of Bio Rad pre-cast CRITERION SDS-gels 8-16%. The IEF strips were fixed in place on the gels by applying a low melting agarose. A second dimensional separation was applied at 200V for about one hour. After running, the gels were carefully removed and placed in a clean tray and washed twice for 20 minutes in 100 ml of pre-staining solution containing 10% methanol and 7% acetic acid.

Staining and Analysis of the 2D Gels

Once the 2D gel patterns of the NAF samples were obtained, the gels were stained with SyproRuby (Bio-Rad Laboratories) and subjected to fluorescent digital image analysis.

The protein patterns of the two breasts for each patient were compared using PDQUEST (Bio-Rad Laboratories) image analysis software. Results of the 2D gel analysis followed by fluorescent staining and image analysis showed that in the 12 unilateral breast cancer patients, differential protein expression patterns were seen when the contralateral breasts were compared. However, in the 4 normal individuals tested, there was a pronounced lack of differential expression in the contralateral breasts.

With identification of different protein expression patterns in the contralateral breasts of unilateral breast cancer patients, these data were used to determine quantitatively which proteins were present in the cancerous breast versus the non-cancerous breasts (indicative of up-regulation of a protein in cancer) as well as which proteins were not present in the cancerous breast as compared to the non-cancerous breast (indicative of down-regulation of a protein in cancer).

To assess the reproducibility of the 2D gels, 75 nanograms of bovine serum albumin (BSA) was run on 9 separate 2D gels. The gels were stained with SyproRuby and the 5 spots that resulted in the BSA region of the gel were then subjected to quantitative analysis using PDQUEST and the Guassian Peak Value method. The results shown in Table 1 illustrate that the electrophoretic patterns were reproducible and independent of the spot amount over the range tested.

Reproducibility of Quantitation in 2D Gels -
PDQuest Peak Value of the Major Components of BSA

| | Spot # | | | | |
|---|---|---|---|---|---|
| Replicate # | 9901 | 9902 | 9904 | 9905 | 9906 |
| 1 | 332 | 1152 | 2612 | 739 | 229 |
| 2 | 246 | 974 | 2694 | 513 | 167 |
| 3 | 336 | 1065 | 2354 | 668 | 225 |
| 4 | 311 | 1272 | 3482 | 713 | 198 |
| 5 | 351 | 1168 | 2724 | 733 | 245 |
| 6 | 268 | 1059 | 2753 | 622 | 184 |
| 7 | 452 | 1630 | 4000 | 946 | 281 |
| 8 | 405 | 1195 | 2752 | 870 | 274 |
| 9 | 258 | 1050 | 2716 | 699 | 189 |
| Avg | 329 | 1174 | 2899 | 723 | 221 |
| Stdev | 68 | 193 | 510 | 127 | 40 |
| CV | 21% | 16% | 18% | 18% | 18% |
| ng/spot | 4.4 | 15.6 | 38.6 | 9.6 | 2.9 |

There were three major protein spots seen in all of the NAF samples taken from both breast of the control or normal individuals that were reduced in most of the NAF samples taken from the breasts that had been diagnosed as cancerous. These Protein spots were isolated and identified as described below.

The Isolation and Identification of the Acetyl-LDL Receptor

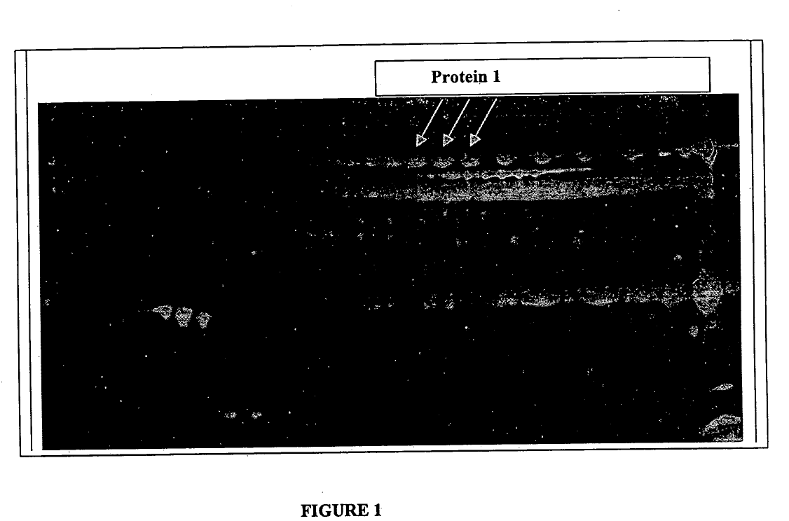

FIG. 1 identifies the three major protein spots (herein referred to as Protein 1) seen in all of the control NAF samples that were reduced in ten of the twelve unilateral breast cancer patients. These protein spots were excised, in-gel digested with trypsin, subjected to mass fingerprinting analysis by matrix-assisted laser desorption ionization-time of flight mass spectrometry (MALDI-TOF MS) and expert database searching.

Mass spectrometry provides a powerful means of determining the structure and identity of complex organic molecules, including proteins and peptides. The unknown compound is bombarded with high-energy electrons causing it to fragment in a characteristic manner. The fragments, which are of varying weight and charge, are then passed through a magnetic field and separated according to their mass/charge ratios. The resulting characteristic fragmentation pattern of the unknown compound is used to identify and quantitate the unknown compound.

MALDI-TOF MS is a type of mass spectrometry in which the analyte substance is distributed in a matrix before laser desorption. The analyte, co-crystallized with a matrix compound, is subjected to pulse UV laser radiation. The matrix, by strongly absorbing the laser light energy, indirectly causes the analyte to vaporize. The matrix also serves as a proton donor and receptor, acting to ionize the analyte in both positive and negative ionization modes. A protein can often be unambiguously identified by a MALDI-TOF MS analysis of its constituent peptides (produced by either chemical or enzymatic treatment of the sample).

Following differential expression analysis, Protein 1 was carefully excised from the gel for identification. Excised gel spots were destained by washing the gel spots twice in 100 mM NH₄HCO₃ buffer, followed by soaking the gel spots in 100% acetonitrile for 10 minutes. The acetonitrile is aspirated, before adding the trypsin solution.

Typically a small volume of trypsin solution (approximately 5-15 μl/ml trypsin) is added to the destained gel spots and incubated at 3 hours at 37° C. or overnight at 30° C. The digested peptides were extracted, washed, desalted and concentrated before spotting the peptide samples onto the MALDI-TOF MS target.

Mass spectral analyses of the digested peptides were performed to identify Protein 1. Those of skill in the art are familiar with mass spectral analysis of digested peptides. The mass spectral analysis was conducted on a MALDI-TOF Voyager DE STR (Applied Biosystems). Spectra were carefully scrutinized for acceptable signal-to-noise ratio (S/N) to eliminate spurious artifact peaks from the peptide molecular weight lists. Both internal and external standards were employed to calibrate any shift in mass values during mass spectroscopic analysis. The external standards were a set of proteins having known molecular weights and known mass/charge ratios in their mass spectrum. A mixture of external standards is placed on the mass spec chip well next to the well that includes an unknown sample. Internal standards are characteristic peaks in the sample spectrum that belong to peptides of the proteolytic enzyme (e.g., trypsin) used to digest the protein spots and extracted along with the digested peptides. Those peaks are used for internal calibration of any deviation of the spectral peaks of the sample.

Corrected molecular weight lists were then subjected to public database searches. The GenBank and dbEST databases maintained by the National Center for Biotechnology Information (hereinafter referred to as the NCBI database) were searched, as well as the SwissProt or Swiss Protein database maintained by ExPasy. Those of skill in the art are familiar with searching databases like the NCBI and SwissProt databases.

The NCBI database search results were displayed according the MOWSE score (a measure of the match probability between the search entry and any proteins identified from the search results). The search results also provided the number of the 94 peptides submitted that were matched and percentage of those peptides matched. The top two matches identified by the NCBI database search were listed as human endothelial cell scavenger receptor precursor (acetyl-LDL receptor) and the human KIAA0149 gene product related to Notch 3. Not only was the MOWSE Score for each of these proteins identical (1.85×10³¹), but also both proteins matched all 94 peptides submitted with a 100% match probability. Furthermore, when the sequence alignment of the human acetyl-LDL receptor was compared with the human Notch 3 protein using the BLOSUM-62 comparison matrix a 99.9% identity of the 830 residues of the two proteins was obtained with a gap frequency of 0.0%. Thus, the best two protein matches identified by the NCBI database (i.e., the acetyl-LDL receptor and the human KIAA0149 gene product related to Notch 3) were assumed to be the same protein, hereinafter referred to simply as the acetyl-LDL receptor. In addition, the Swiss Protein database search identified the same protein as the NCBI database (i.e., the acetyl-LDL receptor) as the closest match to Protein 1.

Further evidence as to the significance of the identification of Protein 1 as the acetyl-LDL receptor is provided in that the third best match identified by the NCBI database was a human unnamed protein with a MOWSE Score of 5.52×10⁵ (as compared to 1.85×10³¹ for AcLDLr/Notch3) and 30 of the 94 peptides matching with a 31% match probability (as compared to a 99.9% match probability for AcLDLr/Notch3). Thus, the identification of Protein 1 as the acetyl-LDL receptor was verified using the analytical tools of proteomic bioinformatics.

The Acetyl-LDL Receptor in Normal and Diseased Breast

The occurrence of acetyl-LDL receptor was quantitated in breast ductal fluid samples collected from both breasts of 12 unilateral breast cancer patients, 4 control or normal women without any known breast disease, and two at-risk subjects (i.e., two mammogram negative women with a history of breast cancer in their family, where the onset of disease in their family members began at the same age as their age when the NAF samples were collected).

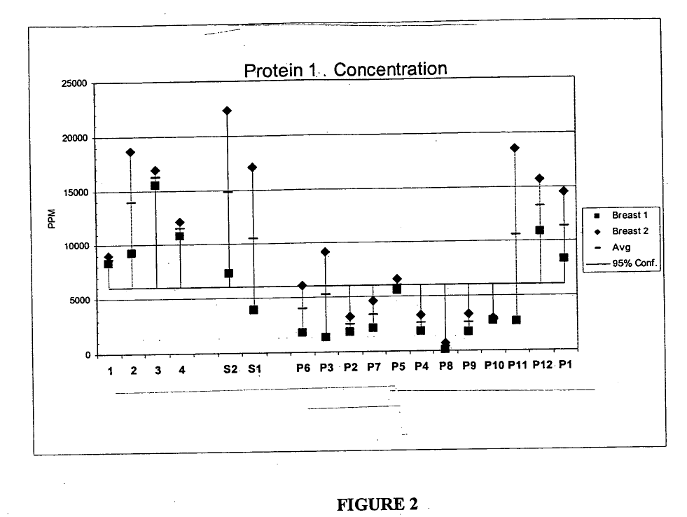

NAF samples were collected from both breasts of four normal women and tested for acetyl-LDL receptor concentration. These eight normal breasts expressed high concentrations of acetyl-LDL receptor as shown in FIG. 2. The eight normal breasts had an average concentration of 12,581 ppm of acetyl-LDL receptor. The acetyl-LDL receptor concentration in the normal breasts ranged from about 8,279 ppm to about 18,669 ppm with a 95% lower confidence limit of 6,073 ppm. The mean concentration of acetyl-LDL receptor in the eight control breasts was 12,581 ppm with a standard deviation of 3,956 ppm. Thus, a normal value of acetyl-LDL receptor protein in control NAF samples was determined to be equal to or more than 8,625 ppm (the mean value minus one standard deviation) or more than or equal to 6,073 ppm (the 95% lower confidence limit of the concentration of the acetyl-LDL receptor in control breasts).

The current use of physical examination, MRI and mammography are useful screening procedures for the early detection of breast cancer, but these methods produce a substantial percentage of false positive and false negative results. In fact it is thought 20% to 25% of women, between 40-49, will have false negative mammographic results leading to a much later than desirable diagnosis of breast cancer. Since more than one out of every ten women will be diagnosed with breast cancer in their life time, it is imperative that new adjunct diagnostic procedures be developed to further enhance breast cancer screening and, thereby, to reduce mortality rates. Since a reduced expression of acetyl-LDL receptor in NAF fluid is a sensitive and highly predictive risk indicator for breast cancer, it is suggested that the accepted normal value of acetyl-LDL receptor protein in control breast ductal fluid be determined very conservatively so that women with low values be monitored more frequently and more intensely, even if the low value seen is statistically within the normal range for control NAF samples.

NAF samples of both breasts of the twelve unilateral breast cancer patients were analyzed for their acetyl-LDL receptor levels. The cancerous breast of the twelve patients had an average acetyl-LDL receptor level of 3,400 ppm with a standard deviation of 3,204 ppm. Ten of the twelve patients had an acetyl-LDL receptor level in their cancerous breasts that was less than the 6,073 ppm that was the lower 95% confidence level of the control breasts. FIG. 2 shows the values for each of the breasts of all twelve patients diagnosed with unilateral breast cancer (shown as P1 to P12 in FIG. 2).

Two of the patients (P1 and P12) had normal levels of acetyl-LDL receptor in both of their breast and could not have been detected by measuring the acetyl-LDL receptor concentration in the NAF sample. The cancerous breasts in the other ten patients had a concentration of acetyl-LDL acetyl receptor that was less than the lower 95% confidence level of the normal breasts, indicating a strong correlation between the reduced acetyl-LDL receptor expression in a NAF sample and the presence of breast disease in the breast from which the sample was taken (i.e., an 83.3% correlation). It is interesting to note that six of the twelve non-cancerous breasts of the patients diagnosed with unilateral breast cancer had less acetyl-LDL receptor than the lower 95% confidence level of normal breasts. This fact indicates that women with reduced levels of acetyl-LDL receptor (i.e., below the 95% confidence limit of normals) are at high risk to develop breast cancer and should be treated as having a pre-cancerous condition and become the object of increased medical surveillance at the very least.

In addition, to the four normal women and twelve women diagnosed with unilateral breast cancer, two at-risk women having a strong familial breast cancer history, but with no evidence of breast disease by mammography or manual breast examination, were investigated for their NAF acetyl-LDL receptor levels. As shown in FIG. 2, there was a large difference in the NAF acetyl-LDL receptor levels between the right and left breasts of these two women (S1 and S2 shown in FIG. 2). The woman, identified in FIG. 2 as S1, had the most profound family history of breast cancer between the two at-risk women. This woman's (S1) mother, grandmother and three maternal great aunts had breast cancer that was diagnosed when these relatives were the same age and the woman was when the NAF sample was collected. The acetyl-LDL receptor level in this woman's (S1) left breast was 3,920 ppm of acetyl-LDL receptor (well below the 95% confidence limit of normals), although her right breast had 17,101 ppm of acetyl-LDL receptor (well above the 95% confidence limit of normals).

To further validate the use of acetyl-LDL receptor as a biomarker for breast disease and particularly for the early detection of breast cancer and for a high risk of developing breast cancer, NAF samples of the right and left breast of this mammogram negative woman (S1) were investigated for the presence of other known breast cancer markers such as HER2/neu. A number of known breast cancer markers were found in the left breast (which had a lower than normal concentration of acetyl-LDL receptor level) and not in the right breast (which had a normal concentration of acetyl-LDL receptor). The specific breast cancer markers detected in the left breast and not in the right breast are listed in Table 2.

Known Breast Cancer Markers Found in the Left Breast
but not in the Right Breast

| Protein Marker | Mass (kDa)/pI | Mr/pI on gel |
|---|---|---|
| 14-3-3 alpha/beta | 27.7/4.79 | 27.7/4.9 |
| 14-3-3 sigma | 27.7/4.65 | 27.3/4.8 |
| 14-3-3 zeta/delta | 27.7/4.78 | 27.5/4.3 |
| Annexin I | 38.8/6.6 | 38.5/6.5 |
| Annexin III | 33.6/5.63 | 36.6/5.9 |
| Annexin V | 31/4.94 | 32.2/4.8 |
| Calreticulin | 55/4.3 | 57.8/4.6 |
| Cathepsin D | 43.2/5.9 | 41.9/5.9 |
| Cytokeratin-K8 | 53.5/5.52 | 52.0/5.5 |
| Cytokeratin K18 | 44.4/5.3 | 46.9/5.3 |
| GST | 24.2/5.6 | 24.6/5.3 |
| HER2/neu | 21.3/6.9 | 23.9/6.8 |
| HSP-27 | 27.1/5.8-6.6 | 27.9/6.5 |
| Maspin | 42.1/5.72 | 41.9/5.9 |
| PCNA | 32/4.57 | 34.1/4.7 |
| PTEN | 47.2/5.9 | 46.9/5.9 |
| Rho GDI | 27.6/4.9 | 25.1/5.0 |

Since S1's right breast exhibited an acetyl-LDL receptor level of a normal breast and the left breast exhibited an acetyl-LDL receptor level of a cancerous breast the finding of the known breast cancer markers in the left breast and not in the right breast was a further validation of the use of acetyl-LDL receptor as a biomarker for the early detection of breast cancer or a high risk of developing breast cancer. Thus, any women having a reduced expression of acetyl-LDL receptor in a NAF sample taken from one or more of her breasts should become the object of increased medical surveillance at the very least.

Acetyl-LDL Receptor Concentrations in the Diagnosis, Prognosis and Therapeutics of Breast Cancer

Currently women are screened for breast cancer using physical examination and mammography. While these methods are useful screening procedures for the early detection of breast cancer, these methods produce a substantial percentage of false positive and false negative results especially in women with dense parenchymal breast tissue. For example, the probability of having a false negative mammogram is 20% to 25% for women between 40-49 and even higher in younger women.

In the United States 15% of all women will be diagnosed with breast cancer during their lifetime. Success in treating breast cancer is dependant upon the early diagnosis of the disease. To date no method for screening women for breast cancer has been totally accurate, so there is a need for new adjunct diagnostic procedures to further enhance cancer screening and, thereby, to reduce mortality rates.

The present invention provides a sensitive method for early detection and diagnosis of breast cancer by assessing the acetyl-LDL receptor concentration in breast nipple aspirate fluid or breast tissue. An acetyl-LDL receptor concentration in nipple aspirate fluid collected from a patient's breast that is significantly below the acetyl-LDL receptor concentration of nipple aspirate fluid samples from normal breasts indicates a strong likelihood of breast cancer or a pre-cancerous condition in the breast having the reduced expression of acetyl-LDL receptor. The average acetyl-LDL receptor value for the ten normal breasts studied was 12,581 ppm with a standard deviation of 3,956 ppm. The 95% lower confidence level of acetyl-LDL receptor for normal breasts was 6,073 ppm. A solid line is drawn across the graph of FIG. 2 representing this 95% confidence limit.

Considering the high probability of obtaining false negative mammographic results, the control population tested may well include some women with a false negative mammographic result. For example, it may be that the normal subject shown in FIG. 2 labeled as (1) is a false negative or has some form of pre-cancerous disease. Despite this known high number of false negative results in breast cancer screening by current methodology, eight NAF samples from the breasts of women with no known breast disease were taken as the control values in this study.

It is readily apparent that all 8 normal breasts are well above this 95% confidence limit, where the lowest acetyl-LDL receptor value of the eight normal breasts is 8,279 ppm. Ten of the twelve breasts diagnosed with breast cancer in patients P1-P12 had acetyl-LDL receptor levels that fell well below the 95% confidence limit of normal breast representing 83.3% of the cancerous breasts. There were, however, two patients P1 and P12 that had acetyl-LDL receptor levels within the normal range resulting in a 16.7% false negative result in the 12 patients tested.

In addition to correctly identifying 10 of the 12 cancerous breasts, the measurement of acetyl-LDL receptor levels indicated breast cancer or a pre-cancerous condition in one high-risk subject (S1) described above and in six of the twelve non-cancerous breasts of the patients diagnosed with unilateral breast cancer. This result highlights the sensitivity of the assay and indicates that women with unilateral breast cancer are at high risk to develop breast cancer in their as yet undiagnosed breast and should become the object of increased medical surveillance and testing.

The acetyl-LDL receptor assay is also useful in assessing the risk of developing breast cancer in one breast versus the other breast by comparing the acetyl-LDL receptor levels in nipple aspirate fluid samples collected from the right and left breast of a patient. By comparing NAF samples collected from both breasts of a patient, the patient's hormonal state and other individual differences are reflected in both breasts. Thus, when the two breasts exhibit widely divergent levels (e.g., when one breast has about 50% or less of acetyl-LDL receptor concentration as the other breast) and the breast with the lower level of acetyl-LDL receptor concentration falls below the mean and one standard deviation of control values (i.e., 8,625 ppm) of acetyl-LDL receptor, the patient is considered at high-risk for the development of breast cancer in the breast having the lower level of acetyl-LDL receptor. Thus, the patient should seek further monitoring for breast cancer.

In one embodiment of the present invention, the acetyl-LDL receptor levels of a breast are obtained by collecting nipple aspirate fluid from the breast, subjecting the NAF to 2D gel electrophoresis; staining the proteins separated by the 2D gel electrophoresis, and quantitating the acetyl-LDL receptor protein spot by the intensity of staining in relation to the intensity of staining as described above. In certain embodiments the first dimensional gel is an isoelectric focusing gel, and the second gel is a denaturing polyacrylamide gradient gel.

The NAF samples may also be subjected to various other techniques known in the art for separating and quantitating proteins. Such techniques include, but are not limited to gel filtration chromatography, ion exchange chromatography, reverse phase chromatography, affinity chromatography, typically in an HPLC or FPLC apparatus, or any of the various centrifugation techniques well known in the art. Certain embodiments would also include a combination of one or more chromatography or centrifugation steps combined via electrospray or nanospray with mass spectrometry or tandem mass spectrometry of the proteins themselves, or of a total digest of the protein mixtures. Certain embodiments may also include surface enhanced laser desorption mass spectromety or tandem mass spectrometry, or any protein separation technique that determines the pattern of proteins in the mixture either as a one-dimensional, two-dimensional, three-dimensional or multi-dimensional pattern or list of proteins present, or list of their post synthetic modification isoforms.

The quantitation of a protein by antibodies directed against that protein are well known in the field. The techniques and methodologies for the production of one or more antibodies to acetyl-LDL receptor are routine in the field and are not described in detail herein.

As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies are generally preferred. However, “humanized” antibodies are also contemplated, as are chimeric antibodies from mouse, rat, or other species, bearing human constant and/or variable region domains, bispecific antibodies, recombinant and engineered antibodies and fragments thereof.

The term “antibody” thus also refers to any antibody-like molecule that has an antigen binding region, and includes antibody fragments such as Fab′, Fab, F(ab′)2, single domain antibodies (DABS), Fv, scFv (single chain Fv), and the like. The techniques for preparing and using various antibody-based constructs and fragments are well known in the art. Means for preparing and characterizing antibodies are also well known in the art (See, e.g., Antibodies: A Laboratory Manual, Cold Spring Harbor Laboratory, 1988; incorporated herein by reference).

Antibodies to the acetyl-LDL receptor may be used in a variety of assays in order to quantitate the acetyl-LDL receptor in a nipple aspirate, or other fluid or tissue sample. Well known methods include immunoprecipitation, antibody sandwich assays, ELISA and affinity chromatography methods that include antibodies bound to a solid support. Such methods also include microarrays of antibodies or proteins contained on a glass slide or a silicon chip, for example.

It is contemplated that arrays of antibodies to acetyl-LDL receptor, or peptides derived from the acetyl-LDL receptor may be produced in an array and contacted with the ductal secretion samples described herein or with the antibodies as appropriate in order to quantitate the acetyl-LDL receptor. The use of such microarrays is well known in the art and is described, for example in U.S. Pat. No. 5,143,854, incorporated herein by reference.

The present invention includes a screening assay for breast cancer based on the down regulation of acetyl-LDL receptor expression. One embodiment of the assay will be constructed with antibodies to acetyl-LDL receptor. One or more antibodies targeted to the acetyl-LDL receptor will be spotted onto a surface, such as a polyvinyl membrane or glass slide. As the antibodies used will each recognize an antigenic determinant of acetyl-LDL receptor, incubation of the spots with patient samples will permit attachment of the acetyl-LDL receptor to the antibody. The acetyl-LDL receptor binding can be reported using any of the known reporter techniques including radioimunoassays (RIA), stains, enzyme-linked immunosorbant assays (ELISA), sandwich ELISAs with a horse radish peroxidase (HRP)-conjugated second antibody also recognizing the acetyl-LDL receptor, the pre-binding of fluorescent dyes to the proteins in the sample, or biotinylating the proteins in the sample and using an HRP-bound streptavidin reporter. The HRP can be developed with a chemiluminescent, fluorescent of colorimetric reporter. Other enzymes such as luciferase or glucose oxidase, or any enzyme that can be used to develop light or color can be utilized at this step.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A biomarker of breast cancer comprising a reduced quantity of an acetyl-LDL-receptor protein in a nipple aspirate fluid sample.

2. A method of using nipple aspirate fluid to diagnose breast cancer, the method comprising:
collecting a nipple aspirate fluid sample from a test subject;
analyzing the nipple aspirate fluid sample for a reduced expression of an acetyl-LDL receptor protein; and
using the expression of the acetyl-LDL receptor protein to diagnose the test subject.

3. The method of claim 2, wherein the diagnosis is an adjunct to at least one other diagnostic test for breast cancer.

4. The method of claim 3, wherein the other diagnostic test is a mammogram or MRI.

5. A method for screening for breast cancer comprising:
obtaining a breast ductal secretion from a patient's breast;
determining a quantity of an acetyl-LDL receptor protein in the patient's breast ductal secretion; and
comparing the quantity of the acetyl-LDL receptor protein in the patient's breast ductal secretion with a normal value of the acetyl-LDL receptor protein in a breast ductal fluid obtained from a set of control breasts;
whereby a reduction in the quantity of the acetyl-LDL receptor protein in the patient's breast ductal secretion to a level less than the normal value of the acetyl-LDL receptor protein in the breast ductal fluid obtained from the set of control breasts is indicative of a cancerous or a pre-cancerous condition in the patient's breast.

6. The method of claim 5, wherein the ductal secretion is obtained by nipple aspiration or ductal lavage.

7. The method of claim 5, wherein the quantity of the acetyl-LDL receptor protein is determined using two-dimensional gel electrophoresis.

8. The method of claim 7, wherein the two-dimensional gel electrophoresis comprises a separation by isoelectric point followed by a separation by molecular weight.

9. The method of claim 7, wherein the protein in the breast ductal secretion is precipitated and the precipitated protein fraction is separated by two-dimensional gel electrophoresis.

10. The method of claim 7, wherein the two-dimensional gel is stained and an intensity of the acetyl-LDL receptor protein staining is used to determine the quantity of the acetyl-LDL receptor protein.

11. The method of claim 5, wherein the quantity of the acetyl-LDL receptor protein is determined using an antibody directed against an antigenic determinant in the acetyl-LDL receptor protein.

12. The method of claim 5, wherein the quantity of the acetyl-LDL receptor protein in the patient's ductal secretion is determined by contacting the ductal secretion with at least one antibody with reactivity to the acetyl-LDL receptor protein.

13. The method of claim 5, wherein the normal value of the acetyl-LDL receptor protein in the breast ductal fluid from the set of control breasts is equal to a mean concentration of the acetyl-LDL receptor protein concentrations in the set of control breasts minus one standard deviation of the mean.

14. The method of claim 5, wherein the normal value of the acetyl-LDL receptor protein in the breast ductal fluid from the set of control breasts is equal to a lower 95% confidence limit in a concentration of the acetyl-LDL receptor protein in the set of control breasts.

15. A method for diagnosing breast cancer comprising:
obtaining a breast ductal secretion from two breasts of a subject;
determining a quantity of an acetyl-LDL receptor protein in the breast ductal secretion of each of the two breasts; and
comparing a concentration of the acetyl-LDL receptor protein in a breast ductal fluid obtained from a set of control breasts with a lowest quantity of the acetyl-LDL receptor protein found in the patient's two breasts.

16. The method of claim 15, wherein a cancerous or pre-cancerous condition in the subject's breast having the lowest quantity of the acetyl-LDL receptor protein is indicated whenever the lowest quantity of the acetyl-LDL receptor protein is at least 50% less than the quantity of the acetyl-LDL receptor protein in the ductal secretion of the other breast and is equal to or less than the mean minus one standard deviation of the acetyl-LDL receptor protein concentration in the set of control breasts.

17. The method of claim 15, wherein the ductal secretion is obtained by nipple aspiration.

18. The method of claim 15, wherein the quantity of acetyl-LDL receptor protein is determined using two-dimensional gel electrophoresis.

19. The method of claim 18, wherein the two-dimensional gel electrophoresis comprises a separation by isoelectric point followed by a separation by molecular weight.

20. The method of claim 18, wherein the quantity of the acetyl-LDL receptor protein is determined using an antibody directed against an antigenic determinant in the acetyl-LDL receptor protein.

21. The method of claim 15, wherein the quantity of the acetyl-LDL receptor protein in the patient's ductal secretion is determined by contacting the ductal secretion with at least one antibody with reactivity to the acetyl-LDL receptor protein.

22. A method for diagnosing breast cancer comprising:
obtaining a nipple aspirate fluid sample from a breast;
separating a protein fraction of the nipple aspirate fluid sample by two-dimensional gel electrophoresis;
determining a quantity of an acetyl-LDL receptor protein in the nipple aspirate fluid sample; and
using the quantity of the acetyl-LDL receptor protein to diagnose breast cancer in the breast.

23. The method of claim 22, further comprising performing an additional diagnostic test for breast cancer.

24. The method of claim 22, wherein the protein fraction is separated by precipitation.

25. The method of claim 24, wherein the precipitated protein fraction is washed with trichoroacetic acid and acetone.

26. The method of claim 22, wherein the quantity of the acetyl-LDL receptor protein is determined using two-dimensional gel electrophoresis.

27. The method of claim 26, wherein the two-dimensional gel electrophoresis comprises a separation by isoelectric point followed by a separation by molecular weight.

28. The method of claim 22, wherein the protein in the breast ductal secretion is precipitated and the precipitated protein fraction is separated by two-dimensional gel electrophoresis.

29. The method of claim 26, wherein the two-dimensional gel is stained and an intensity of the acetyl-LDL receptor protein staining is calculated.

30. The method of claim 22, wherein the quantity of acetyl-LDL receptor protein is determined using an antibody directed against an antigenic determinant in the acetyl-LDL receptor protein.

31. The method of claim 22, wherein the quantity of the acetyl-LDL receptor protein in the breast nipple aspirate fluid sample is determined by contacting the two-dimensional gel with at least one antibody with reactivity to the acetyl-LDL receptor protein.

32. A method for diagnosing breast cancer comprising:
obtaining a breast ductal secretion from a breast;
determining a quantity of an acetyl-LDL receptor protein in the breast ductal secretion using an antibody reactive with an antigenic determinant in the acetyl-LDL receptor protein; and
using the quantity of the acetyl-LDL receptor protein to diagnose breast cancer in the breast.

33. The method of claim 32, further comprising performing an additional diagnostic test for breast cancer.

34. The method of claim 32, wherein a plurality of antibodies reactive with an antigenic determinant in the acetyl-LDL receptor protein are used to determine the quantity of the acetyl-LDL receptor protein in the breast ductal secretion.

35. The method of claim 32, wherein the antibody is a monoclonal antibody.

36. The method of claim 32, wherein the antibody is a chimeric antibody.

37. The method of claim 32, wherein the antibody is an antiserum, an Fab antibody fragment, a monoclonal antibody, a chimeric antibody, a IgG immunogobulin, an IgM immunoglobulin, or a combination of the same.

38. The method of claim 32, wherein the amount of antibody reacted with the acetyl-LDL receptor protein is reported using an radioimmunoassay, an enzyme-linked immunosorbent assay, or a sandwich enzyme-linked immunosorbent assay.

39. The method of claim 32, wherein an amount of the antibody reacted with the acetyl-LDL receptor protein is reported using a horseradish peroxidase reporter, a strepavidin reporter, a fluorescent reporter, a chemiluminescent reporter, a colorimetric reporter, or a combination of the same.
